(19) United States
(12) Patent Application Publication
(10) Pub. No.: US 2024/0242804 A1
(43) Pub. Date: Jul. 18, 2024

(54) VIDEOCONFERENCE DATA BASED MACHINE LEARNING MODEL TO IDENTIFY A CALCULATED RISK ASSESSMENT TO GENERATE A CARE PLAN FOR A COLLABORATIVE CARE MODEL

(71) Applicant: Bend Health, Inc., Madison, WI (US)

(72) Inventors: Kurt Roots, Madison, WI (US); Monika Roots, Madison, WI (US)

(21) Appl. No.: 18/411,497

(22) Filed: Jan. 12, 2024

Related U.S. Application Data
(60) Provisional application No. 63/438,645, filed on Jan. 12, 2023.

(51) Int. Cl. G16H 20/00; G06Q 30/04; G16H 10/60; G16H 50/20; G16H 50/30

(52) U.S. Cl. CPC G16H 20/00; G06Q 30/04; G16H 10/60; G16H 50/20; G16H 50/30

(57) ABSTRACT

In at least one example, a method for utilizing an enhanced collaborative care model includes receiving videoconference data from a videoconference between a patient and a first provider, incorporating the interview data with the videoconference data, entering the videoconference data and incorporated interview data into an electronic health record (EHR) associated with the patient, calculating risk assessments for the patient based on the interview data, providing the interview data and calculated risk assessments to a machine learning model configured to generate a care plan for the patient based on the calculated risk assessments and the interview data, determining a plurality of additional providers for the patient based on the generated care plan for the patient, and generating a care plan schedule based on a schedule of the plurality of additional providers, a schedule of the patient, and a timeline of the generated care plan for the patient.

### TECHNICAL FIELD

The present disclosure relates to devices and systems to improve enhanced collaborative medical care. Such techniques can be particularly useful to centrally treat patients requiring specialty care, generate and communicate care plans across different provider types, schedule patients with available health care providers that are able provide the services of the care plans, and provide billing information to insurance organizations.

### BACKGROUND

Healthcare is a critical aspect of human well-being, and access to timely and effective medical services is essential for maintaining and improving overall health. However, healthcare systems worldwide face challenges such as increasing healthcare costs, the burden of chronic diseases, and limited access to medical facilities and specialists, particularly in rural or underserved areas.

Remote health monitoring and intervention systems have emerged as a promising solution to address these challenges. These systems leverage technology to enable healthcare providers to remotely monitor the vital signs, health conditions, and compliance of patients, reducing the need for frequent in-person visits and hospitalizations.

Traditional methods of healthcare monitoring often involve periodic clinic visits, which may not capture real-time data or provide immediate intervention when health issues arise. Additionally, patients with chronic conditions or those requiring continuous monitoring, such as the young or elderly or individuals with specific medical conditions, face limitations in their ability to access timely care.

### SUMMARY OF THE DISCLOSURE

The present disclosure is directed to using improvements in machine learning technology to predict a property of a product (e.g., quality, effectiveness, defects, failures, etc.). Image based visual analysis utilizing a machine learning model can be utilized to monitor and analyze a production process to predict a property of the product based on production analysis data. The image based visual analysis can utilize a machine learning model to interpret and extract data from captured images by identifying relevant patterns and features within the images. These features can include, but are not limited to: edges, textures, shapes, colors, and/or other visual attributes. A prediction of a property of the product can be received from the machine learning model and used to adjust the production process or to determine whether to reject the product.

In some aspects, the techniques described herein relate to a method, including: receiving videoconference data from a videoconference between a patient and a first provider; incorporating the interview data with the videoconference data; entering the videoconference data and incorporated interview data into an electronic health record (EHR) associated with the patient; calculating risk assessments for the patient based on the interview data; providing the interview data and calculated risk assessments to a machine learning model configured to generate a care plan for the patient based on the calculated risk assessments and the interview data; determining a plurality of additional providers for the patient based on the generated care plan for the patient; and generating a care plan schedule based on a schedule of the plurality of additional providers, a schedule of the patient, and a timeline of the generated care plan for the patient.

In some aspects, the techniques described herein relate to a method, wherein the interview data includes assessments, digital biomarkers, risk trajectory, caregiver reported information and patient reported information. In some aspects, the techniques described herein relate to a method, wherein the digital biomarkers are extracted from the videoconference data.

In some aspects, the techniques described herein relate to a method, further including: receiving additional interview data and additional videoconference data from an additional videoconference between one of the plurality of additional providers and the patient; calculating updated risk assessments for the patient based on the additional interview data; and providing the additional interview data and updated risk assessments to the machine learning model configured to generate an updated care plan for the patient based on the updated risk assessments and the additional interview data.

In some aspects, the techniques described herein relate to a method, further including altering the care plan schedule based on the updated care plan for the patient. In some aspects, the techniques described herein relate to a method, further including generating an assessment score prediction for the patient based on a comparison between the interview data and the additional interview data. In some aspects, the techniques described herein relate to a method, wherein the assessment score prediction is an exponential moving average at a particular time. In some aspects, the techniques described herein relate to a method, further including calculating a bend risk score (BRS) for the patient based on the interview data and additional interview data, wherein the BRS is a prediction of risk for the patient at a point in time during the care plan.

In some aspects, the techniques described herein relate to a machine-readable medium, storing machine-readable instructions which, when executed by a processor of a device, cause the processor to: receive videoconference data from a videoconference between a patient and a provider; incorporate the interview data with the videoconference data; enter the videoconference data and incorporated interview data into an electronic health record (EHR) associated with the patient that includes historical interview data from historical videoconference data; calculate risk assessments of the patient based on the interview data and the historical interview data associated with the patient, wherein the historical interview data is extracted from the EHR; generate an assessment score prediction value for the patient based on trend data associated with the historical interview data and interview data from the videoconference between the patient and the provider; provide the assessment score prediction value to a machine learning model configured to generate a care plan for the patient based on the assessment score prediction value of a patient with a patient background similar to the patient; determine a plurality of additional providers for the patient based on the generated care plan for the patient; and generate a care plan schedule based on a schedule of the plurality of additional providers, a schedule of the patient, and a timeline of the generated care plan for the patient.

In some aspects, the techniques described herein relate to a machine-readable medium, including instructions to normalize the assessment score prediction value. In some aspects, the techniques described herein relate to a machine-readable medium, including instructions to categorize the patient based on the normalized assessment score prediction value. In some aspects, the techniques described herein relate to a machine-readable medium, including instructions to provide the normalized assessment score prediction value to the machine learning model to update the care plan.

In some aspects, the techniques described herein relate to a machine-readable medium, wherein the care plan includes a quantity of time to spend with each of the plurality of additional providers and a licensure requirement for each of the plurality of additional providers. In some aspects, the techniques described herein relate to a machine-readable medium, including instructions to calculate a bend risk score (BRS) for the patient based on a plurality of factors associated with the patient including: age of the patient; gender of the patient; primary condition of the patient, medication prescribed to the patient; Medicaid or insurance status of the patient; Social Determinants of Health (SDoH) value of the patient; adherence to care of the patient; adherence to modules of the care plan of the patient; quantity of self-reported doses of medication missed by the patient; and quantity of monthly count of Rapid Support Team (RST) escalations.

In some aspects, the techniques described herein relate to a system, including: an electronic health record (EHR) database; a scheduling database; a billing database; a medication database; a device configured to: receive videoconference data from a videoconference between a patient and a provider; incorporate the interview data with the videoconference data; enter the videoconference data and incorporated interview data into an EHR, stored in the EHR database, associated with the patient that includes historical interview data from historical videoconference data stored in the EHR database; calculate risk assessments of the patient based on the interview data and the historical interview data associated with the patient, wherein the historical interview data is extracted from the EHR database; generate an assessment score prediction value for the patient based on trend data associated with the historical interview data and interview data from the videoconference between the patient and the provider; provide the assessment score prediction value to a machine learning model configured to generate a care plan for the patient based on the assessment score prediction value of a patient with a patient background similar to the patient; determine a plurality of additional providers for the patient based on the generated care plan for the patient; generate a care plan schedule based on a schedule of the plurality of additional providers, a schedule of the patient, and a timeline of the generated care plan for the patient; store the care plan at the scheduling database; calculate a bill for the patient based on the interview data; and store the bill for the patient at the billing database.

In some aspects, the techniques described herein relate to a system, wherein the EHR database, scheduling database, medication database, and billing database each include separate security configurations. In some aspects, the techniques described herein relate to a system, wherein the bill includes a combination of billing data associated with the patient from the plurality of additional providers, wherein the billing data includes CPT codes, minutes accrued, practitioner qualifications, and care length of the patient.

In some aspects, the techniques described herein relate to a system, wherein the device is configured to: send the combination of billing data to a health care provider of the patient to be reviewed prior to sending to an insurance provider of the patient. In some aspects, the techniques described herein relate to a system, wherein the device is configured to: receive payment associated with the bill directly from the insurance provider of the patient. In some aspects, the techniques described herein relate to a system, wherein the care plan defines a plurality of modules from a Learning Resource Center (LRC) that includes interactive lesson plans and materials that when completed by the patient are stored in the EHR database.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1A illustrates an example of a method of an enhanced collaborative care model.

FIG. 1B illustrates an example of a method of an enhanced collaborative care model.

FIG. 2 is one example diagram illustrating a system of an enhanced collaborative care model.

FIG. 3 illustrates an example of a method for dynamic assessments delivered to a caregiver and/or patient at baseline and monthly of an enhanced collaborative care model.

FIG. 4 illustrates an example of a method of scheduling for an enhanced collaborative care model.

FIG. 5 illustrates an example of a method of care delivery for an enhanced collaborative care model.

FIG. 6 illustrates an example of a method of billing for an enhanced collaborative care model.

FIG. 7 illustrates an example of a method of an enhanced collaborative care model.

FIG. 8 illustrates an example of a machine readable medium for an enhanced collaborative care model.

FIG. 9 illustrates an example of a device for an enhanced collaborative care model.

### DETAILED DESCRIPTION

The present disclosure relates to methods and devices for improving enhanced collaborative care models, which may utilize machine learning models to generate care plans and determine a scheduling plan for patients that utilize a plurality of care providers. An example of a machine learning model is an ANN. The ANN can provide learning by forming probability weight associations between an input and an output. The probability weight associations can be provided by a plurality of nodes that comprise the ANN. The nodes together with weights, biases, and/or activation functions can be used to generate an output of the ANN based on the input to the ANN. A plurality of nodes of the ANN can be grouped to form layers of the ANN.

A machine learning model can be a function or equation for identifying patterns in data. A machine learning module can be a plurality of machine learning models utilized together to identify patterns in data. In a specific example, a machine learning module can be organized as a neural network. A neural network can include a set of instructions that can be executed to recognize patterns in data. Some neural networks can be used to recognize underlying relationships in a set of data in a manner that mimics the way that a human brain operates. A neural network can adapt to varying or changing inputs such that the neural network can generate an acceptable result in the absence of redesigning the output criteria.

An enhanced collaborative care model can utilize a plurality of parties to provide health care resources to patients. The enhanced collaborative care model can include parties such as physicians, psychologists, psychiatrists, mental health coaches, insurance companies, schedulers, among other professionals that can collaborate to provide resources to patients. These enhanced collaborative care models can be difficult to organize and utilize since each patient is unique with unique needs, unique schedules, and/or a unique set of symptoms and progression through a care plan. In this way, it can be difficult to provide the services a particular patient needs at a plurality of stages of care for the patient.

Innovation in the care delivery model for mental healthcare has become a much more prevalent discussion because of a dramatic increase in demand for services coupled with a shortage of licensed experts. This generally has been the promise of telehealth, which seamlessly connects patient demand to provider supply in a frictionless way through video conferencing and technology. While helping reduce barriers to care, the traditional care model in mental health which pairs one patient to one therapist or one patient to one psychiatrist doesn't go far enough because of the extreme shortage of providers. There are not enough providers to deliver 1:1 synchronous telehealth sessions given all of the patients that need care.

This disclosure describes a specific data driven system that relies on measurement and algorithmic methods to facilitate, and drive continued scalable and evidence-based care interventions through enhanced collaborative care teams. Machine learning models and other systems can be utilized to optimize the care provided to each of a plurality of different patients utilizing a plurality of different providers. In this way, the patients are able to receive the medical care they need over a period of time when they need the medical care.

As used herein, the singular forms “a”, “an”, and “the” include singular and plural referents unless the content clearly dictates otherwise. Furthermore, the word “may” is used throughout this application in a permissive sense (e.g., having the potential to, being able to), not in a mandatory sense (e.g., must). The term “include,” and derivations thereof, mean “including, but not limited to.”

As will be appreciated, elements shown in the various embodiments herein can be added, exchanged, and/or eliminated so as to provide a number of additional embodiments of the present disclosure. In addition, as will be appreciated, the proportion and the relative scale of the elements provided in the figures are intended to illustrate certain embodiments of the present invention and should not be taken in a limiting sense.

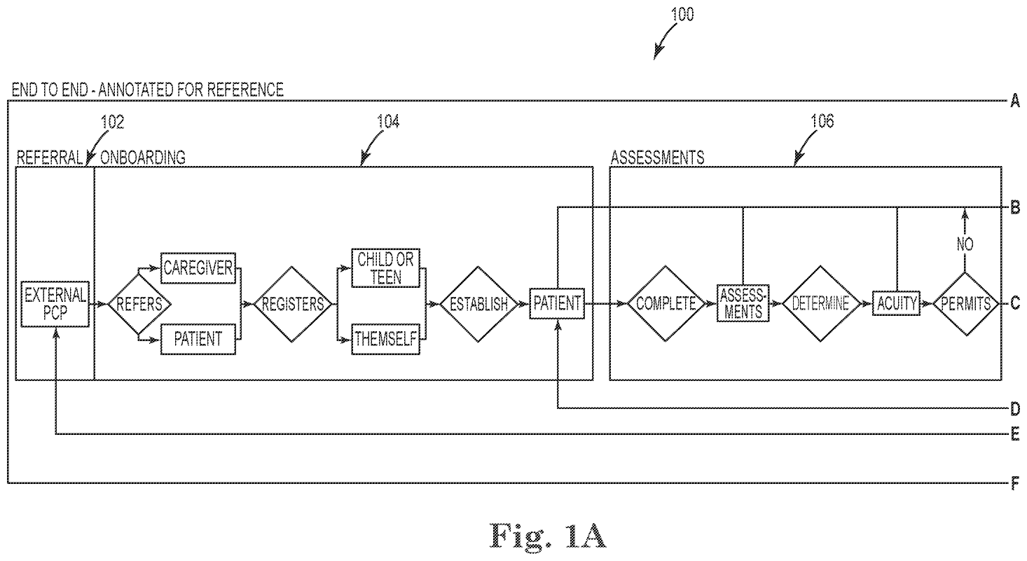

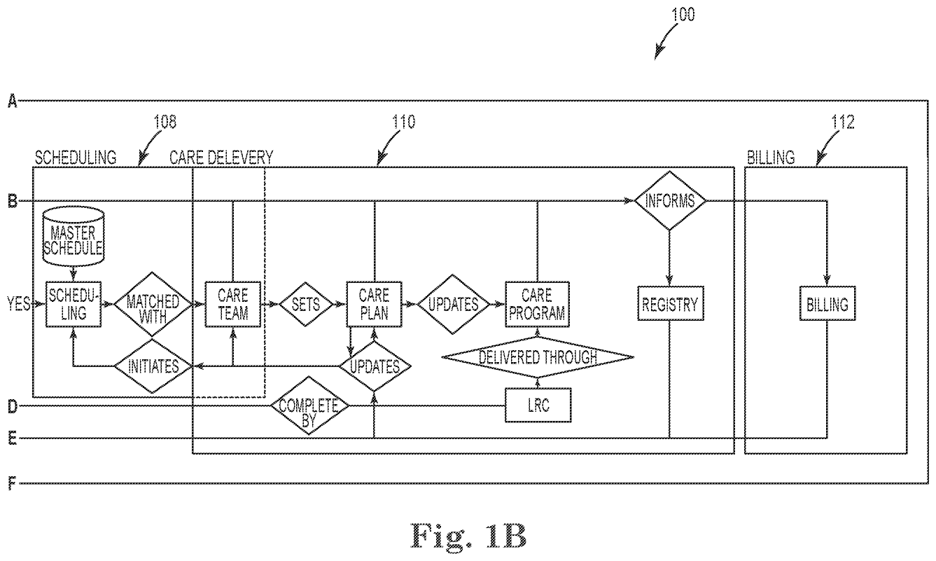

FIG. 1A and FIG. 1B illustrates an example of a method 100 of an enhanced collaborative care model. The complete end-to-end method 100 that drives care to patients is described in FIG. 1A and FIG. 1B collectively. FIG. 1A describes a sophisticated healthcare management system that uses interactive and adaptive questionnaires, audio and video data analysis, and comprehensive electronic health record (EHR) creation to tailor and coordinate clinical pathways for patients, enhancing personalized care and provider collaboration. Once through onboarding and into care, the method 100 drives the scalable and evidence-based care delivery by algorithmically triggering human-based interventions.

The method 100 can include a referral process 102. In some examples, the referral process 102 can refer to a process of receiving patient referrals from physicians or other health care providers. The referral process 102 can be part of the health care provider's evaluation of the patient and the patient can be referred to the enhanced collaborative care model of providers based on a plurality of factors. Once the referral process 102 is completed. The method 100 can move to the onboarding process 104. The onboarding process 104 can include an analysis of the patient to determine, among other things: whether the referral is for a patient or caregiver, whether the referral is for a child/teenager or for themselves, whether to establish the referral as a patient to proceed with the method 100.

The method 100 can include an assessment process 106. The assessment process 106 can include a plurality of assessments and acuity of the patient. As described further herein, the plurality of assessments can include, but are not limited to: an assessment score prediction (ASP), a bend risk score (BRS), a Patient Health Questionnaire-9 (PHQ-9) score, and/or a Generalized Anxiety Disorder 7 (GAD-7) score. As used herein, the PHQ-9 score can be calculated utilizing a part of the larger Patient Health Questionnaire (PHQ) family of assessments, which are designed to screen and monitor various mental health conditions in clinical and research settings. The PHQ-9 specifically focuses on depression and consists of nine questions or items that individuals rate based on their experiences over the past two weeks. Each of the nine items in the PHQ-9 is related to common symptoms of depression, and respondents are asked to rate the frequency with which they have experienced these symptoms.

As used herein, the GAD-7 score is designed to assess the severity of generalized anxiety disorder symptoms in individuals. It is commonly utilized by healthcare professionals, psychologists, and researchers to screen for, diagnose, and monitor the symptoms of generalized anxiety disorder (GAD). The GAD-7 consists of seven questions or items that individuals rate based on their experiences over the past two weeks. The GAD-7 serves several purposes, including screening for generalized anxiety disorder, assessing the severity of anxiety symptoms, monitoring changes in symptoms over time, and aiding healthcare professionals in making treatment decisions. It is often used in primary care settings, mental health clinics, and research studies to assess and track anxiety symptoms in individuals. A higher GAD-7 score may suggest a need for further evaluation and potentially treatment for generalized anxiety disorder.

The method 100 can continue as illustrated in FIG. 1B. Based on the plurality of assessments, the patient can either be referred out to other resources or the patient can proceed to the scheduling process 108. The scheduling process 108 can include generating a schedule for the patient to connect with health care providers based on the availability of the healthcare providers and patient, as well as the qualifications of the healthcare providers and assessments of the patient. The scheduling database can be utilized to determine the generated schedule for the patient. For example, the scheduling database can include real time scheduling information for the plurality of providers and real time scheduling information for the patient.

The method 100 can include a care delivery process 110. The care delivery process 110 can include determining the care team, determining the care plan, receiving updates during the care plan, generating a care program with an LRC, generating a registry, among other aspects of a remote care program. Upon completion of one or more of the care delivery process 110, the method 100 can include a billing process 112. As described further herein, the billing process 112 can include billing patients, insurance companies, and/or health care providers that initiated the original referral of the patient.

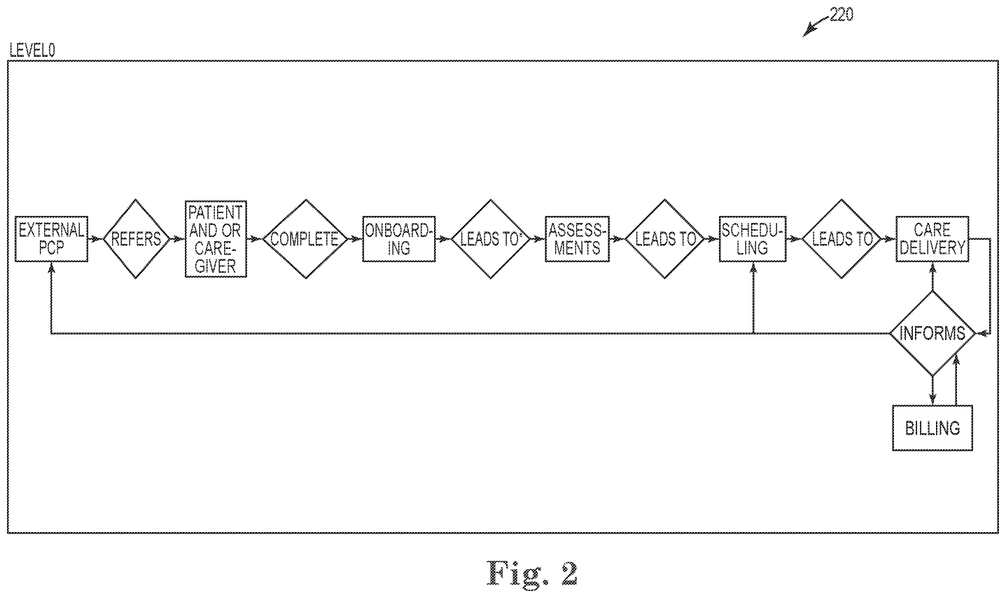

FIG. 2 is one example diagram illustrating a system 220 of an enhanced collaborative care model. FIG. 2 describes the components used in collaborate care model. The overall system 220 is initiated through a Primary Care Physician (PCP) Referral who is external to the system 220. This initiates the onboarding and assessment process (e.g., onboarding process 104 as illustrated in FIG. 1, assessment process 106 as illustrated in FIG. 1, etc.) for the caregiver and/or patient. Scheduling then occurs, but the system 220 is flexible enough to allow for scheduling to occur prior to taking assessments and recording video and audio. Care delivery or Treatment then begins (e.g., care delivery process 110 as illustrated in FIG. 1, etc.). Scheduling and care delivery are then updated iteratively as necessary based on treatment progress and information from caregiver and/or patient.

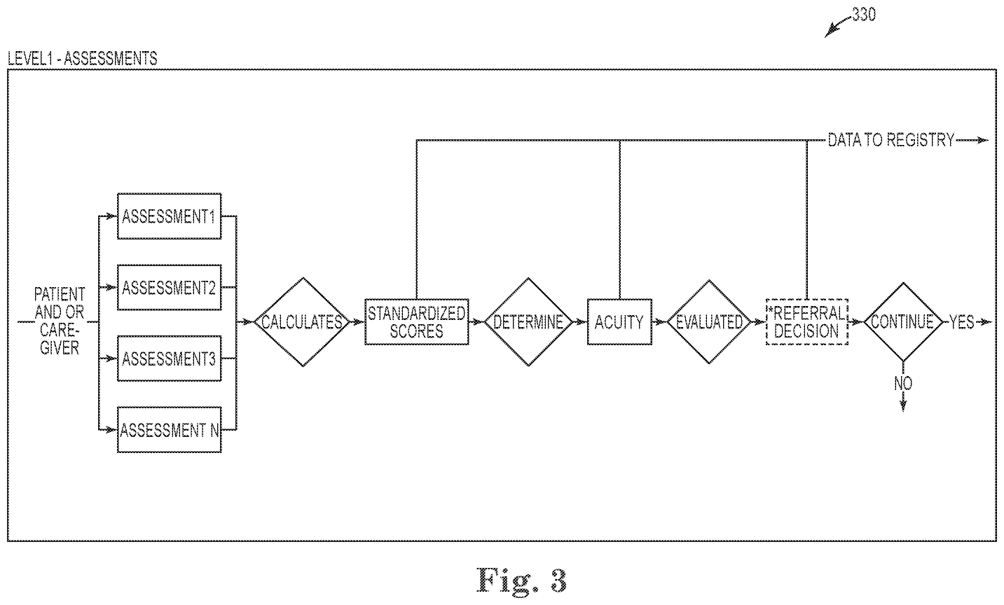

FIG. 3 illustrates an example of a method 330 for dynamic assessments delivered to a caregiver and/or patient at baseline and monthly of an enhanced collaborative care model. FIG. 3 reviews the dynamic assessments delivered to the caregiver and/or patient at baseline and then monthly. Two examples of standardized assessments use to screen for mental health conditions include the PHQ-9 (Patient Health Questionnaire-9) and GAD-7 (General Anxiety Disorder-7), which are standardized screening tools used to assess the severity of depression and anxiety, respectively. Although PHQ-9 and GAD-7 are described as assessments herein, other types of assessments can be utilized without departing from the disclosure.

The PHQ-9 is a 9-item self-report questionnaire that scores each of the DSM-IV criteria for depression on a scale from 0 (not at all) to 3 (nearly every day). It's used to identify the presence and severity of depressive symptoms within the past two weeks, with the total score ranging from 0 to 27. Breakdown of the scoring calculation, which indicates severity or acuity: 0-4: Minimal or no depression, 5-9: Mild depression, 10-14: Moderate depression, 15-19: Moderately severe depression, 20-27: Severe depression. The higher the score, the more severe the depression is considered to be. This tool can help guide clinical decisions regarding further evaluation and treatment. GAD-7: The GAD-7 is a 7-item self-report questionnaire that scores common anxiety symptoms. Each item is scored similarly to the PHQ-9, from 0 (not at all) to 3 (nearly every day), reflecting the frequency of symptoms experienced over the last two weeks. The total score for the GAD-7 ranges from 0 to 21. Breakdown of the scoring calculation: 0-4: Minimal anxiety, 5-9: Mild anxiety, 10-14: Moderate anxiety, and 15-21: Severe anxiety. Like the PHQ-9, the GAD-7 score can indicate the need for further evaluation and can be used to monitor symptom severity over time. Both tools are widely used in both clinical and research settings due to their reliability and validity for screening. The method 330 implements these plus additional standard screening instruments. The method 330 implements thresholds which allows for the referral decision to be configurable and accept lower or higher acuity scores before either entering care or continuing with care.

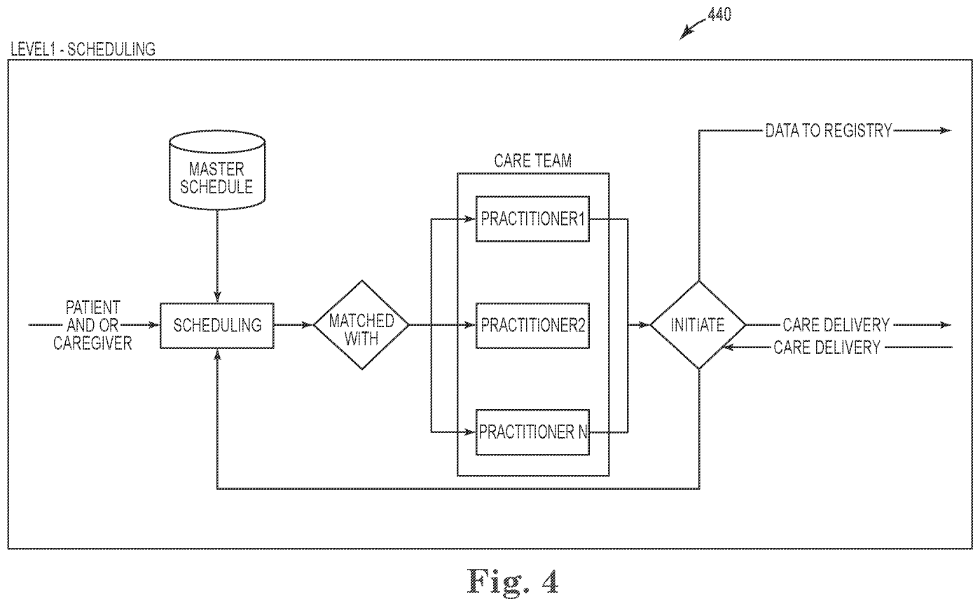

FIG. 4 illustrates an example of a method 440 of scheduling for an enhanced collaborative care model. FIG. 4 describes the process flow for scheduling, which matches a caregiver and/or patient to one or more members of the care team (e.g., plurality of care providers, etc.). Scheduling can be for an intake to start care or follow-up visits after care is established. Scheduling an intake can start with a Behavioral Care Manager (BCM) who reviews assessments, digital biomarkers, risk trajectory, and caregiver or patient reported information as part of thorough evaluation process which establishes the right level of care and required care plan.

Care plans establish the structure of the care team and determine the type of care programs to deliver. Care plans include services like: neuropsychological testing, mental health coaching, mental health coaching with the addition of psychiatry, coaching with the addition of therapy, coaching with the addition of therapy and psychiatry, and/or virtual intensive outpatient. As used herein, mental health coaching refers to a form of supportive and guidance-oriented coaching focused on helping individuals improve their mental well-being, emotional resilience, and overall psychological health. It is not a substitute for therapy or clinical treatment but complements traditional mental health services by offering practical strategies, motivation, and encouragement to individuals seeking to enhance their mental health.

As used herein, psychiatry refers to medical care provided by a psychiatrist. Psychiatry is a branch of medicine that focuses on the diagnosis, treatment, and prevention of mental disorders and emotional issues. Psychiatrists are medical doctors who specialize in this field and are trained to evaluate both the physical and psychological aspects of mental health. In some examples, the psychiatrists can work to provide medication management and/or psychotherapy. As used herein, therapy can refer to counseling provided by psychologists, social workers, and/or counselors.

Care programs can be based on cognitive-behavioral therapy (CBT) and/or dialectical behavior therapy (DBT) principles. CBT is a widely recognized and evidence-based approach to psychotherapy used in the field of mental health. CBT is based on the premise that our thoughts, feelings, and behaviors are interconnected, and it focuses on helping individuals understand and change unhelpful thought patterns and behaviors to improve their mental well-being. DBT is a specialized form of CBT to help individuals with borderline personality disorder (BPD). Since its inception, DBT has been adapted and expanded to address a broader range of mental health conditions and challenges. It is known for its emphasis on acceptance and change, as well as its structured and skills-based approach.

Care programs are stratified by age, condition or concern, and acuity can involve different evidence-base techniques and methods based on CBT and DBT principles, which are designed to be completed over the course of four to six months. These evidence-based interventions are delivered by different care teams and are designed to be age and condition appropriate. Caregivers and or Patients can engage with this interactive content and materials during session visits or outside of Care Team visits through the Learning Resource Center (LRC). The LRC can include a plurality of learning modules that can be utilized by patients during their care plan to help with the CBT and/or DBT techniques.

Care Programs exist for things like: Stress, Anxiety, Back to School Transitions, Substance Use, and post-traumatic stress disorder (PTSD). Care Teams are a collection of practitioners, which may include one: BCM, Mental Health Coach, Therapist (may or may not be assigned), Psychiatrist (may or may not be assigned) This spectrum of Practitioners will be scheduled based after the intake by the BCM and may be adjusted at any time depending on conditions and acuity needs that arise from Care Delivery and information presented by the Caregiver and/or Patient. Each practitioner type has a different frequency for delivering care based on the care plan selection. The scheduling interval is based both the Care Plan and the Care Plan Cycle, which is defined as the minimum threshold of visits to be delivered by Practitioner within a 28 to 30 day span.

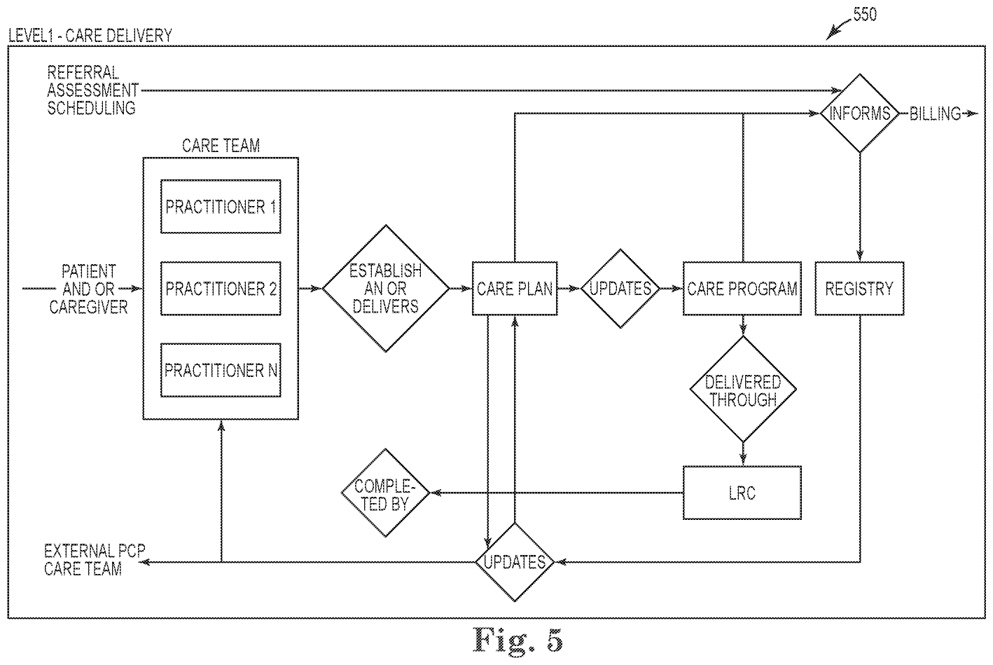

FIG. 5 illustrates an example of a method 550 of care delivery for an enhanced collaborative care model. FIG. 5 describes the care delivery process. The caregiver and/or patient, along with one practitioner from the care team, have a virtual face-to-face video visit. Visits that occur update the patient's medical record which is part of the EHR, with the progress towards completing the overall care plan, but also the care program, along with updating the medical record. Care plans may be updated based on needs of the caregiver and/or patient and changes to symptoms, acuity, and any additional information. Visits may leverage the learning resource center (LRC), which contains the interactive lesson plans and materials. All of this information also updates the registry.

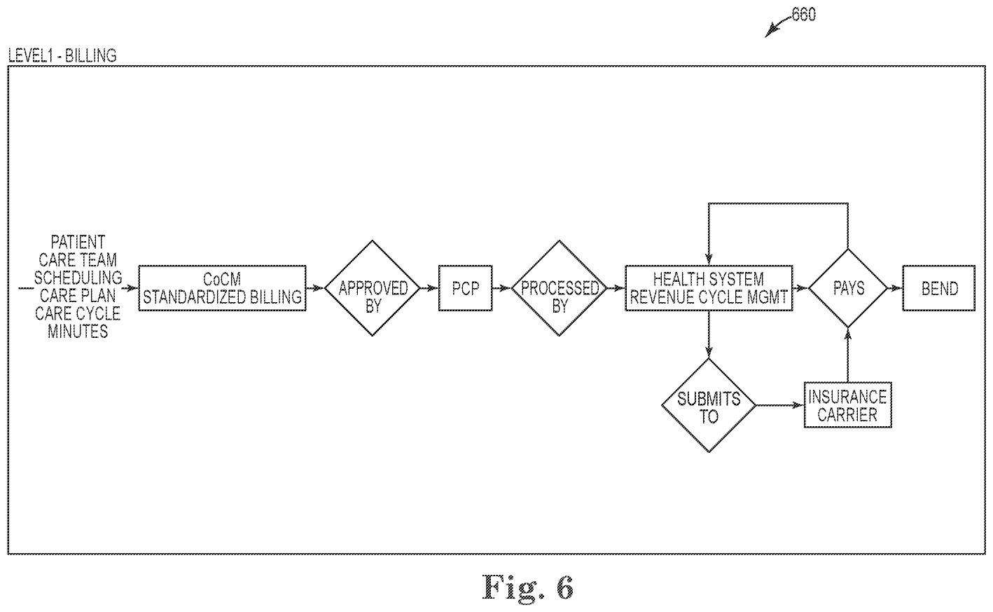

FIG. 6 illustrates an example of a method 660 of billing for an enhanced collaborative care model. FIG. 6 describes the Billing process. The method 660 for billing follows the standard policies and procedures outlined by The Centers for Medicare & Medicaid Services (CMS) for delivering care under the collaborate care models Current Procedural Terminology (CPT) Codes. This means: standards such as the CPT codes, minutes accrued, practitioner qualifications, care length, etc. are utilized to coalesce the care information from each Patient in care at the end of a monthly care cycle. This data is rolled-up into a super bill and delivered to the partnering health system. The primary care provider (PCP) approves the minutes of care delivered by an organization implementing the devices systems described herein for each referred patient. The health system Revenue Cycle Management (RCM) department reviews and submits the approved minutes of care to the insurance carrier for reimbursement. The health system then pays the mental health delivery organization a contracted rate for the delivered care. The health system is compensated directly by the insurance carrier for the submitted claims. The Registry contains the same information that's available in the EHR, but in a shared portal that is accessible between the organization referring patients and the organization delivering care. This information includes, but is not limited to the following information: Referral Information, Bend Risk Score (BRS), Assessment Score Prediction (ASP), Assessment scores, Care adherence (CA), Program adherence (PA), Medication adherence (MA), Rapid Support Team (RST), Care Team, and Scheduling.

In some examples, the assessment score prediction (ASP) can be calculated utilizing Equation 1. The sequence of assessment scores for each standardized assessment captured monthly per patient is S1, S2, S3, . . . , St where each Si represents the score for each assessment at time i and t can be any positive integer representing the total number of scores up to the current period. The formula of Equation 1 is: EMAt=α·St+(1−α)·EMAt−1. Where: EMAt I is the exponential moving average at time t, St is the score at time t, a is the smoothing factor, which is a number between 0 and 1, and EMAt−1 is the exponential moving average of the previous time period.

The Bend Risk Score (BRS) is a prediction of risk for a given Patient at a point in time during their treatment. The BRS signals to the Care Team the risk level of a Patient and allows them to drill into components of the score to take the necessary actions to lower their risk. The BRS is represented mathematically as follows:
Ŷₜ=f(Xage, Xgender, Xcondition, Xmedication, Xmedicaid, Xsdoh, XCAdherence, XP Adherence, XMAdherence, XRST, EMAhistorical,θ)
Where: Ŷᵢ=the predicted score at time/and Xage, Xgender, Xcondition, Xmedication, Xmedicaid, Xsdoh, XC Adherence, XP Adherence, XMAdherence, XRST, EMAhistorical are independent factors described below in Table 1.

TABLE 1

| Factor | Name | Type | Description | Calculation |
|---|---|---|---|---|
| Xage | Age | Decimal | Age of Patient | Self-reported |
| Xgender | Gender | Categorical | Gender of Patient | Self-reported |
| Xcondition | Condition | Categorical | Primary mental health | ICD10 Code |
| | | | condition | |
| Xmedication | Medication | Binary | Flag indicating whether | 1 = yes |
| | | | Patient is taking a | 0 = no |
| | | | psychotropic medication. | |
| Xmedicaid | Medicaid | Binary | Flag representing Patient | 1 = yes |
| | | | covered by Medicaid 1 = | 0 = no |
| | | | yes and 0 = no | |
| Xsdoh | SDoH | Numeric | Social Determinants of | Standardized |
| | | | Health (SDoH) Screener | Calculation |
| XCAdherence | Care | Decimal | Patient adherence to care | CAdherence = |
| | Adherence | | | V_{attended}/Vₜₒₜₐₗ |
| | | | | V_{attended} = visits |
| | | | | Vₜₒₜₐₗ = total visits. |
| XPAdherence | Program | Decimal | Patient adherence of | PAdherence = |
| | Adherence | | modules completed in care | M_{completed}/Mₜₒₜₐₗ |
| | | | programs | M_{completed} = count |
| | | | | of mods |
| | | | | completed |
| | | | | M_{possible} = total |
| | | | | mods |
| XMAdherence | Medication | Decimal | Monthly count of self- | XMAdherence = |
| | Adherence | | reported doses missed | M_{occurrence}/Mₘₒₙₜₕₛ |
| | | | | M_{occurrence} = count |
| | | | | of missed doses |
| | | | | Mₘₒₙₜₕₛ = months |
| | | | | in care |
| XRST | RST | Decimal | Monthly count of Rapid | XRST = R_{occurrence}/ |
| | | | Support Team (RST) | Rₘₒₙₜₕₛ |
| | | | escalations | R_{occurrence} = count |
| | | | | of RST |
| | | | | escalations |
| | | | | Rₘₒₙₜₕₛ = months |
| | | | | in care |
| EMAhistorical | Assessment | Decimal | Exponential Moving | Previously |
| | Score | | Average of historical | defined |
| | Prediction | | assessment scores | |

In Table 1, F represents the machine learning model, which allows for a range of possible machine learning models, like a linear regression, decision tree, or a more complex model like a neural network. Θ represents the parameters or coefficients of the model, which are learned from historical data during the training phase.

The BRS and ASP is configurable to be able to segment Patients with higher risk from lower risk, and those with higher predictive scores compared to lower predictive scores. This is based on a segmenting formula used in the Registry. In a specific example, the values are normalized relative to their scale. For example, Min-Max Scaling is commonly used to achieve this objective. This is represented as follows: Xnorm=X−Xmin/Xmax−Xmin. In this example, the values are then segmented into four categories of risk: no risk, low risk, moderate risk, and/or high risk. In this example, R is the risk score, intervals can be flexibly defined as follows: No Risk: R is in the range [0, A), Low Risk: R is in the range [A, B), Moderate Risk: R is in the range [B, C), and/or High Risk: R is in the range [C, D). In this example, A, B, C, and D are specific numerical values that define the boundaries of each risk category. These values satisfy the conditions: 0≤A<B<C≤D.

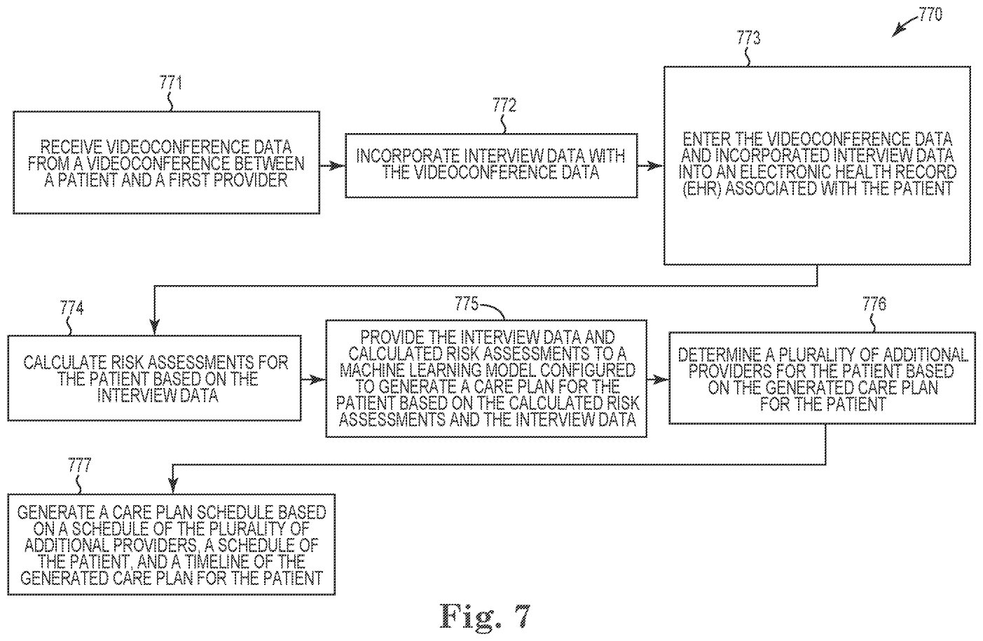

FIG. 7 illustrates an example of a method 770 for executing an enhanced collaborative care model. The method 770 can be performed by processing logic that can include hardware (e.g., processing device, circuitry, dedicated logic, programmable logic, microcode, hardware of a device, integrated circuit, etc.), software (e.g., instructions run or executed on a processing device), or a combination thereof. Although shown in a particular sequence or order, unless otherwise specified, the order of the processes can be modified. Thus, the illustrated embodiments should be understood only as examples, and the illustrated processes can be performed in a different order, and some processes can be performed in parallel. Additionally, one or more processes can be omitted in various embodiments. Thus, not all processes are required in every embodiment. Other process flows are possible.

At 771, the method 770 can be executed to receive videoconference data from a videoconference between a patient and a first provider. As described herein, the patient can be a human user who is attempting to receive medical service and a provider can be a human user who is attempting to provide medical services or other services to the patient. In some examples, the first provider is a BCM or provider that is performing an onboarding process with the patient. In some examples, the videoconference between the patient and the first provider can be a teleconference that allows video and audio of the meeting between the first provider and the patient to be captured and/or saved as videoconference data.

In this way, the videoconference data can be further analyzed and utilized to extract different biomarkers associated with the patient's reactions, answers, bodily reactions, among other features that can be identified from the audio or video. In some examples, the videoconference data can be provided to a device or server when the videoconference between the patient and the first provider is completed.

At 772, the method 770 can incorporate interview data with the videoconference data. In some examples, the interview data includes assessments, digital biomarkers, risk trajectory, caregiver reported information and patient reported information. In these examples, the digital biomarkers can be extracted from the videoconference data. The assessments can include the survey answers and/or biomarkers during the conducted surveys (e.g., PHQ-9, GAD-7, etc.). The interview data can also include notes from the first provider that can be utilized to assess the patient.

At 773, the method 770 can be executed to enter the videoconference data and incorporated interview data into an electronic health record (EHR) associated with the patient. As described herein, the videoconference data can be incorporated with the interview data to allow the combined data to be uploaded or entered into the EHR associated with the patient. In this way, the progression of the patient can be monitored over the course of the care plan.

At 774, the method 770 can be executed to calculate risk assessments for the patient based on the interview data. The risk assessments can be systematic evaluations conducted by mental health professionals to identify and manage potential risks, especially those related to self-harm, harm to others, or worsening mental health conditions. These assessments play a crucial role in ensuring the safety and well-being of individuals who may be struggling with mental health issues. The risk assessments can be part of the onboarding process as described herein.

At 775, the method 770 can be executed to provide the interview data and calculated risk assessments to a machine learning model configured to generate a care plan for the patient based on the calculated risk assessments and the interview data. As described herein, a machine learning model, such as an ANN or other type of large language model, can be trained to generate a care plan for a particular patient based on the calculated risk assessment, patient information, interview data, and/or other factors described herein that are captured through the assessment process.

As used herein, training the machine learning model can include altering the weighting vectors based on historical information and/or outcomes. In this way, the machine learning model can generate a new care plan for a particular patient based on input information (e.g., assessment information, risk assessments, age, gender, etc.). This information can be provided to the machine learning model and the machine learning model can generate a specific care plan for the patient.

At 776, the method 770 can be executed to determine a plurality of additional providers for the patient based on the generated care plan for the patient. As described herein, the plurality of additional providers can be coaches, physicians, psychiatrists, caregivers, social workers, among other medical professionals that can be utilized to provide mental health services.

At 777, the method 770 can be executed to generate a care plan schedule based on a schedule of the plurality of additional providers, a schedule of the patient, and a timeline of the generated care plan for the patient. The care plan schedule can include a plurality of time lines for completing particular mental health modules or scheduled dates to meet with one or more of the plurality of additional providers. In this way, the care plan schedule can consider the plurality of factors that can affect an availability of the patient and/or the plurality of providers. In addition, the care plan schedule can allow the patient to have sufficient time to complete the mental health modules prior to meeting with a particular health care provider.

In some examples, the method 770 can be performed a plurality of times with a particular patient. For example, the patient can have a plurality of assessments over the course of a care plan to determine the progress of the patient. In some examples, the method 770 can be executed to receive additional interview data and additional videoconference data from an additional videoconference between one of the plurality of additional providers and the patient, calculate updated risk assessments for the patient based on the additional interview data, and provide the additional interview data and updated risk assessments to the machine learning model configured to generate an updated care plan for the patient based on the updated risk assessments and the additional interview data. In this way, the care plan and/or care plan schedule can be continuously updated.

In these examples, the method 770 can be executed to alter the care plan schedule based on the updated care plan for the patient. In some examples, the method 770 can be executed to generate an assessment score prediction for the patient based on a comparison between the interview data and the additional interview data. In these examples, the assessment score prediction is an exponential moving average at a particular time. As described herein, the assessment score prediction can be based on predictive models and algorithms to estimate or predict an individual's mental health assessment scores or outcomes based on various factors, data, and information. These predictive models can be used to assist mental health professionals in making informed decisions about diagnosis, treatment planning, and intervention strategies.

In some examples, the method 770 can be executed to calculate a bend risk score (BRS) for the patient based on the interview data and additional interview data, wherein the BRS is a prediction of risk for the patient at a point in time during the care plan. The BRS is described herein and can be utilized to identify a level of risk associated with a particular patient. The level of risk can refer to a level of risk to self-harm of the patient or other negative outcomes of the patient.

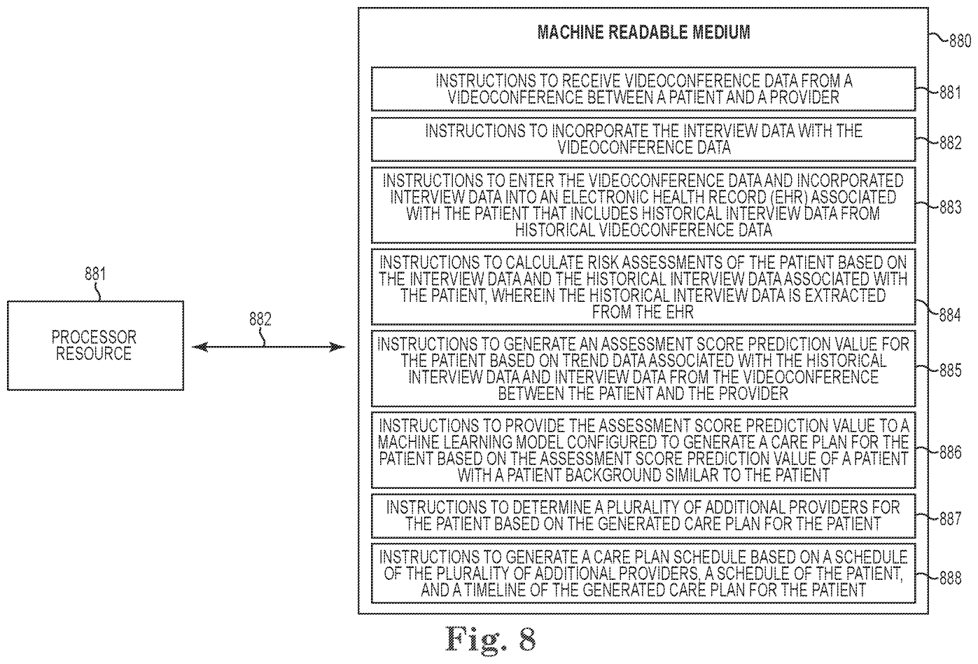

FIG. 8 illustrates an example of a machine readable medium 880 for executing an enhanced collaborative care model. The machine readable medium 880 can be communicatively connected to a processor resource 881 by a communication path 882. In some examples, a communication path 882 can include a wired or wireless connection that can allow communication between devices and/or components within a single device. As used herein, the processor resource 881 can include, but is not limited to: a central processing unit (CPU), an application specific integrated circuit (ASIC), a field programmable gate array (FPGA), a metal-programmable cell array (MPCA), a semiconductor-based microprocessor, or other combination of circuitry and/or logic to orchestrate execution of instructions 881, 882, 883, 884, 885, 886, 887, 888. In a specific example, the processor resource 881 utilizes a non-transitory computer-readable medium storing instructions 881, 882, 883, 884, 885, 886, 887, 888, that, when executed, cause the processor resource 881 to perform corresponding functions.

The machine readable medium 880 may be electronic, magnetic, optical, or other physical storage device that stores executable instructions. Thus, a non-transitory machine-readable medium (MRM) (e.g., machine readable medium 880) may be, for example, a non-transitory MRM comprising Random-Access Memory (RAM), read-only memory (ROM), an Electrically Erasable Programmable ROM (EEPROM), a storage drive, an optical disc, and the like. The machine readable medium 880 may be disposed within a controller and/or computing device. In this example, the executable instructions 881, 882, 883, 884, 885, 886, 887, 888, can be “installed” on the device. Additionally, and/or alternatively, the machine readable medium 880 can be a portable, external, or remote storage medium, for example, which allows a computing system to download the instructions 881, 882, 883, 884, 885, 886, 887, 888, from the portable/external/remote storage medium. In this situation, the executable instructions may be part of an “installation package”.

The machine readable medium 880 includes instructions 881 to receive videoconference data from a videoconference between a patient and a provider. As described herein, videoconference data refers to the digital information exchanged during a videoconference or video call. This data encompasses various types of audio, video, and content-sharing data transmitted over the internet or another network connection. The videoconference data can be utilized to extract information from the audio, video, or other content from the interview between the patient and the provider. For example, biomarkers can be extracted from the videoconference data after the videoconference is completed. The biomarkers can be defined as biological molecules, substances, or characteristics that can be objectively measured and evaluated as indicators of normal biological processes, pathogenic processes, or pharmacological responses to therapeutic interventions.

The machine readable medium 880 includes instructions 882 to incorporate the interview data with the videoconference data. In some examples, the interview data can be data that is collected or analysis from the videoconference data. For example, the interview data can include the biomarker data extracted from the videoconference data. In other examples, the interview data can be results of a test given during the interview. Other examples of interview data can be included to further describe the interview between the patient and the provider.

The machine readable medium 880 includes instructions 883 to enter the videoconference data and incorporated interview data into an electronic health record (EHR) associated with the patient that includes historical interview data from historical videoconference data. The machine readable medium 880 includes instructions 884 to calculate risk assessments of the patient based on the interview data and the historical interview data associated with the patient, wherein the historical interview data is extracted from the EHR.

In some embodiments, the machine readable medium 880 can include instructions to normalize the assessment score prediction value. Normalizing assessment score prediction values can be a preprocessing step in machine learning and statistical modeling to ensure that the values fall within a consistent and standardized range. Normalization helps prevent certain features or variables from dominating the prediction process due to their larger scales. In these examples, the machine readable medium 880 can include instructions to categorize the patient based on the normalized assessment score prediction value. In these examples, the machine readable medium 880 can include instructions to provide the normalized assessment score prediction value to the machine learning model to update the care plan. Since the values are normalized, the categorization and/or updated care plan can be generated by the machine learning model.

The machine readable medium 880 includes instructions 885 to generate an assessment score prediction value for the patient based on trend data associated with the historical interview data and interview data from the videoconference between the patient and the provider. As used herein, trend data refers to an analysis of a plurality of data over time to determine a trend in the data. For example, the trend data can refer to a trend in assessment scores of a particular patient over a period of time. This trend data can be helpful to determine progress in a positive or negative direction, identify what types of treatment may be working, and/or what types of medication are working.

The machine readable medium 880 includes instructions 886 to provide the assessment score prediction value to a machine learning model configured to generate a care plan for the patient based on the assessment score prediction value of a patient with a patient background similar to the patient. In some examples, the patient background can include the age, sex, diagnosis information, and/or prior treatments of the patient. In this way, a similar patient may not have the exact same background, but may include similar features such as being close in age, the same sex, and/or have other factors that are similar (e.g., similar family background, etc.). In some examples, the care plan includes a quantity of time to spend with each of the plurality of additional providers and a licensure requirement for each of the plurality of additional providers.

The machine readable medium 880 includes instructions 887 to determine a plurality of additional providers for the patient based on the generated care plan for the patient. As described herein, the plurality of additional providers can be a care team for the patient based on the plurality of assessment factors of the patient. In this way, the care team can have the credentials and schedule availability to provide the services associate with the care plan.

The machine readable medium 880 includes instructions 888 to generate a care plan schedule based on a schedule of the plurality of additional providers, a schedule of the patient, and a timeline of the generated care plan for the patient. As described herein, the care plan schedule can include a timeline for completion of particular care modules of an LRC, completion of a particular number of sessions or meetings with particular care providers, and/or other goals or tasks to be completed over a period of time.

In some examples, the machine readable medium 880 can include instructions to calculate a bend risk score (BRS) for the patient based on a plurality of factors associated with the patient including: age of the patient; gender of the patient; primary condition of the patient, medication prescribed to the patient; Medicaid status of the patient; Social Determinants of Health (SDoH) value of the patient; adherence to care of the patient; adherence to modules of the care plan of the patient; quantity of self-reported doses of medication missed by the patient; and quantity of monthly count of Rapid Support Team (RST) escalations.

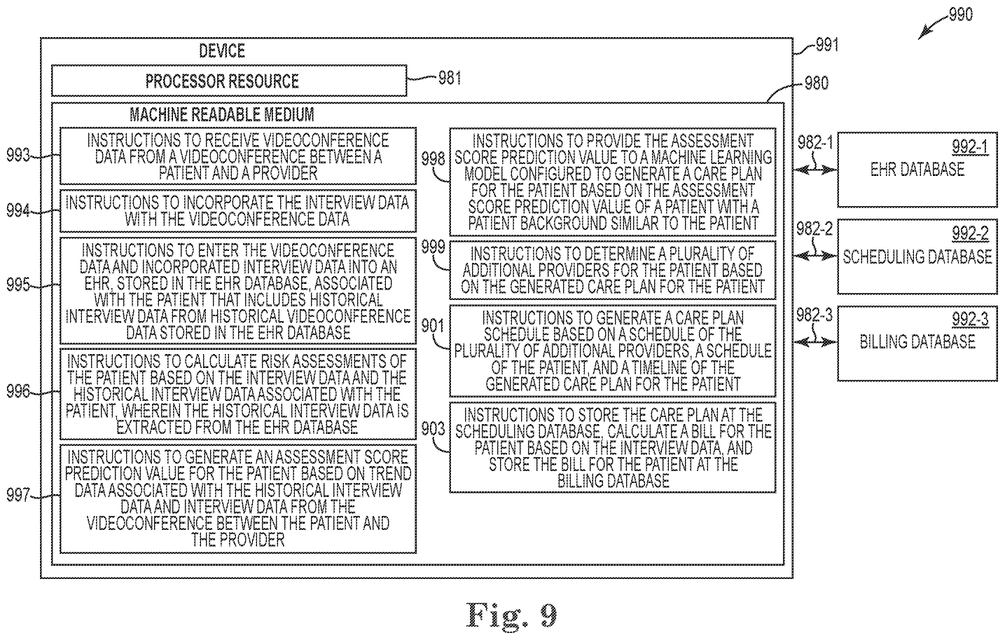

FIG. 9 illustrates an example of a device 901 for executing an enhanced collaborative care model. In some examples, the device 901 is a computing device that includes a processor resource 981 and a machine readable medium 980 to store instructions 993, 994, 995, 996, 997, 998, 999, 901, 903 that are executed by the processor resource 981 to perform particular functions. FIG. 9 illustrates how a computing device can execute instructions to perform functions described herein. The device 901 can be a machine learning model operating on a computing device.

The device 901 can be communicatively coupled to: an EHR database 992-1 through a first communication path 982-1, a scheduling database 992-2 through a second communication path 982-2, and/or a billing database 992-3 through a third communication path 982-3. In some examples, the EHR database 992-1, scheduling database 992-2, and billing database 992-3 each include separate security configurations such that portions of the plurality of providers may be able to access one or more of the databases, but not others. For example, a particular provider may be able to access the scheduling databased 992-2, but not have the credentials to access the billing database 992-3. This can provide confidentiality for patients and providers while also allowing for collaboration between the plurality of providers to provide adequate care for the patient.

The device 901 includes instructions 993 stored by the machine readable medium 980 that is executed by the processor resource 981 to receive videoconference data from a videoconference between a patient and a provider. The device 901 includes instructions 994 stored by the machine readable medium 980 that is executed by the processor resource 981 to incorporate the interview data with the videoconference data. As described herein, the videoconference data can be combined with the interview data to allow access to data associated with a particular meeting between the patient and the provider. In addition, the combined data can be utilized to help identify data trends, which can be very important when updating a care plan.

The device 901 includes instructions 995 stored by the machine readable medium 980 that is executed by the processor resource 981 to enter the videoconference data and incorporated interview data into an EHR, stored in the EHR database 992-1, associated with the patient that includes historical interview data from historical videoconference data stored in the EHR database 992-2. The device 901 includes instructions 996 stored by the machine readable medium 980 that is executed by the processor resource 981 to calculate risk assessments of the patient based on the interview data and the historical interview data associated with the patient, wherein the historical interview data is extracted from the EHR database 992-1. The historical interview data can be interview data and/or videoconference data associated with a previously conducted interview with the patient. In this way, a current interview can be compared to previous interviews.

The device 901 includes instructions 997 stored by the machine readable medium 980 that is executed by the processor resource 981 to generate an assessment score prediction value for the patient based on trend data associated with the historical interview data and interview data from the videoconference between the patient and the provider. The device 901 includes instructions 998 stored by the machine readable medium 980 that is executed by the processor resource 981 to provide the assessment score prediction value to a machine learning model configured to generate a care plan for the patient based on the assessment score prediction value of a patient with a patient background similar to the patient.

The device 901 includes instructions 999 stored by the machine readable medium 980 that is executed by the processor resource 981 to determine a plurality of additional providers for the patient based on the generated care plan for the patient. The device 901 includes instructions 901 stored by the machine readable medium 980 that is executed by the processor resource 981 to generate a care plan schedule based on a schedule of the plurality of additional providers, a schedule of the patient, and a timeline of the generated care plan for the patient. In some examples, the care plan defines a plurality of modules from a Learning Resource Center (LRC) that includes interactive lesson plans and materials that when completed by the patient are stored in the EHR database.

As described herein, a Learning Resource Center (LRC) is a facility or organization that provides a wide range of resources, tools, and support services to enhance learning, research, and professional development in the field of mental health. These centers aim to empower mental health professionals, students, and the general public by offering access to valuable information and educational materials. For example, an LRC can include e-learning platforms and courses focused on mental health topics. These may include webinars, video lectures, and interactive modules that allow individuals to expand their knowledge and skills in areas like therapy techniques, counseling, and mental health advocacy.

The device 901 includes instructions 903 stored by the machine readable medium 980 that is executed by the processor resource 981 to store the care plan at the scheduling database 992-2, calculate a bill for the patient based on the interview data, and store the bill for the patient at the billing database 992-3. In some examples, the bill includes a combination of billing data associated with the patient from the plurality of additional providers, wherein the billing data includes CPT codes, minutes accrued, practitioner qualifications, and care length of the patient. In some examples, the device 901 can be configured to send the combination of billing data to a health care provider of the patient to be reviewed prior to sending to an insurance provider of the patient. In some embodiments, the device 901 can be configured to receive payment associated with the bill directly from the insurance provider of the patient.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Various advantages of the present disclosure have been described herein, but embodiments may provide some, all, or none of such advantages, or may provide other advantages.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A method for generating a care plan for a collaborative care model, comprising:
receiving videoconference data from a videoconference between a patient and a first provider;
incorporating interview data with the videoconference data;
entering the videoconference data and incorporated interview data into an electronic health record (EHR) associated with the patient;
calculating risk assessments for the patient based on the interview data;
providing the interview data and calculated risk assessments to a machine learning model configured to generate a care plan for the patient based on the calculated risk assessments and the interview data;
determining a plurality of additional providers for the patient based on the generated care plan for the patient; and
generating a care plan schedule based on a schedule of the plurality of additional providers, a schedule of the patient, and a timeline of the generated care plan for the patient.

2. The method of claim 1, wherein the interview data includes assessments, digital biomarkers, risk trajectory, caregiver reported information and patient reported information.

3. The method of claim 2, wherein the digital biomarkers are extracted from the videoconference data.

4. The method of claim 1, further comprising:
receiving additional interview data and additional videoconference data from an additional videoconference between one of the plurality of additional providers and the patient;
calculating updated risk assessments for the patient based on the additional interview data; and
providing the additional interview data and updated risk assessments to the machine learning model configured to generate an updated care plan for the patient based on the updated risk assessments and the additional interview data.

5. The method of claim 4, further comprising altering the care plan schedule based on the updated care plan for the patient.

6. The method of claim 4, further comprising generating an assessment score prediction for the patient based on a comparison between the interview data and the additional interview data.

7. The method of claim 6, wherein the assessment score prediction is an exponential moving average at a particular time.

8. The method of claim 4, further comprising calculating a bend risk score (BRS) for the patient based on the interview data and additional interview data, wherein the BRS is a prediction of risk for the patient at a point in time during the care plan.

9. A machine-readable medium, storing machine-readable instructions which, when executed by a processor of a device, cause the processor to:
receive videoconference data from a videoconference between a patient and a provider,
incorporate interview data with the videoconference data;
enter the videoconference data and incorporated interview data into an electronic health record (EHR) associated with the patient that includes historical interview data from historical videoconference data;
calculate risk assessments of the patient based on the interview data and the historical interview data associated with the patient, wherein the historical interview data is extracted from the EHR;
generate an assessment score prediction value for the patient based on trend data associated with the historical interview data and interview data from the videoconference between the patient and the provider,
provide the assessment score prediction value to a machine learning model configured to generate a care plan for the patient based on the assessment score prediction value of a patient with a patient background similar to the patient;
determine a plurality of additional providers for the patient based on the generated care plan for the patient; and
generate a care plan schedule based on a schedule of the plurality of additional providers, a schedule of the patient, and a timeline of the generated care plan for the patient.

10. The machine-readable medium of claim 9, comprising instructions to normalize the assessment score prediction value.

11. The machine-readable medium of claim 10, comprising instructions to categorize the patient based on the normalized assessment score prediction value.

12. The machine-readable medium of claim 11, comprising instructions to provide the normalized assessment score prediction value to the machine learning model to update the care plan.

13. The machine-readable medium of claim 9, wherein the care plan includes a quantity of time to spend with each of the plurality of additional providers and a licensure requirement for each of the plurality of additional providers.

14. The machine-readable medium of claim 9, comprising instructions to calculate a bend risk score (BRS) for the patient based on a plurality of factors associated with the patient including: age of the patient; gender of the patient; primary condition of the patient; medication prescribed to the patient; Medicaid or insurance plan status of the patient; Determinants of Health (SDoH) value of the patient; adherence to care of the patient; adherence to modules of the care plan of the patient; quantity of self-reported doses of medication missed by the patient; and quantity of monthly count of Rapid Support Team (RST) escalations.

15. A system, comprising:
an electronic health record (EHR) database;
a scheduling database;
a billing database;
a medication database;
a device configured to:
receive videoconference data from a videoconference between a patient and a provider;
incorporate interview data with the videoconference data;
enter the videoconference data and incorporated interview data into an EHR, stored in the EHR database, associated with the patient that includes historical interview data from historical videoconference data stored in the EHR database;
calculate risk assessments of the patient based on the interview data and the historical interview data associated with the patient, wherein the historical interview data is extracted from the EHR database;
generate an assessment score prediction value for the patient based on trend data associated with the historical interview data and interview data from the videoconference between the patient and the provider;
provide the assessment score prediction value to a machine learning model configured to generate a care plan for the patient based on the assessment score prediction value of a patient with a patient background similar to the patient;
determine a plurality of additional providers for the patient based on the generated care plan for the patient;
generate a care plan schedule based on a schedule of the plurality of additional providers, a schedule of the patient, and a timeline of the generated care plan for the patient;
store the care plan at the scheduling database;
calculate a bill for the patient based on the interview data; and
store the bill for the patient at the billing database.

16. The system of claim 15, wherein the EHR database, scheduling database, medication database, and billing database each include separate security configurations.

17. The system of claim 15, wherein the bill includes a combination of billing data associated with the patient from the plurality of additional providers, wherein the billing data includes CPT codes, minutes accrued, practitioner qualifications, and care length of the patient.

18. The system of claim 15, wherein the device is configured to: send the combination of billing data to a health care provider of the patient to be reviewed prior to sending to an insurance provider of the patient.

19. The system of claim 18, wherein the device is configured to: receive payment associated with the bill directly from the insurance provider of the patient.

20. The system of claim 15, wherein the care plan defines a plurality of modules from a Learning Resource Center (LRC) that includes interactive lesson plans and materials that iteratively update and store progress data in the EHR when completed by the patient.
